(19) United States
(12) Patent Application Publication
(10) Pub. No.: US 2022/0151552 A1
(43) Pub. Date: May 19, 2022

(54) Method to Time Medication and Other Therapies According to Individual Circadian Time

(71) Applicant: Timeshifter, Inc., Southampton, NY (US)

(72) Inventors: Mickey Beyer-Clausen, Southampton, NY (US); Steven Lockley, Somerville, MA (US); Fredric Maxik, Cocoa Beach, FL (US)

(73) Assignee: Timeshifter, Inc., Southampton, NY (US)

(21) Appl. No.: 16/949,843

(22) Filed: Nov. 17, 2020

(51) Int. Cl. A61B 5/00

(52) U.S. Cl. CPC A61B 5/4848; A61B 5/4812; A61B 5/4836; A61B 5/0022; A61B 5/742; A61B 5/0059; A61B 5/7405

(57) ABSTRACT

A method of determining and administering therapies responsive to a circadian rhythm of a user including receiving an indication of a therapy to be administered to the user, identifying one or more periods during a circadian cycle during which administration of the therapy is preferred, defining a circadian administration time, receiving an indication of a normal circadian rhythm of the user. determining the position of the circadian administration time within the normal circadian rhythm of the user, defining a user circadian administration time, determining a time of day corresponding to the user circadian administration time, defining a clock administration time, and at least one of providing an indication to the user to administer the therapy at the clock administration time and administering the therapy to the user at the clock administration time.

### FIELD OF THE INVENTION

The present invention relates to systems and methods for timing medication and other therapy administration according to a patient's circadian time.

### BACKGROUND OF THE INVENTION

The twenty four-hour central circadian clock, located in the suprachiasmatic nuclei (SCN) in the hypothalamus in the brain, controls the daily timing of multiple brain and body systems including sleep, alertness and performance, metabolism, reproductive rhythms, and immune function, among many others. In addition to this central or ‘master’ circadian clock in the brain, other parts of the brain and peripheral organs and tissues, such as the lungs, liver, heart, pancreas and kidneys, also generate circadian rhythms and work synchronously with the SCN to control and fine-tune local circadian rhythms, such as twenty-four hour rhythms in cardiac, liver, or lung function.

Given the ubiquity of circadian processes, there is increased interest in how to time the administration of various therapies, such as the administration of medication, vaccines, and other pharmaceutical products, and other therapies such as chemotherapy and surgery, to an individual's internal circadian clock, rather than generic timing according to clock time, or without consideration of circadian time.

There is wide variability between individuals in the circadian phase of central and peripheral clocks that ranges from five hours when individuals are studied under highly controlled conditions, to as much as seven to nine hours in people with sleep disorders (e.g., insomnia) or living under irregular schedules, and up to twelve hours in shift workers or after trans-meridian travel (jetlag). This means that a therapy given at 8 a.m. clock time in two individuals might be as much as twelve hours different in circadian time, the equivalent of taking the drug at 8 p.m. in biological time. Several therapies have been shown to vary in their efficacy and safety depending on the time of day which likely vastly underestimates the potential difference in circadian time. Timing therapies based on individual circadian times has enormous potential to improve drug efficacy, for example, inducing the same benefit but with a lower dose, and safety, for example. reducing potentially harmful side-effects or toxicity.

Applicant has previously developed a method for estimating circadian time in order to time ‘chronobiotic’ treatments, treatments that vary in their effectiveness or effect of the circadian clock based in the circadian time of administration, to address jetlag as reflected in U.S. patent application Ser. No. 16/432,544 titled Method and System for Generating and Providing Notifications for a Circadian Shift Protocol filed Jun. 5, 2019, the content of which is incorporated by reference. The method described therein estimates an individual's circadian time based on sleep patterns and chronotype (whether one is a morning type, evening type or neither) in order to time light, light avoidance or melatonin administration, among other activities, to shift the individual's circadian system more quickly than typical approaches (e.g., taking sleeping pills or stimulants, which do not reset the circadian clock). In this invention, a modified algorithm is applied to estimate individual circadian time in order to time the administration of therapies, that would result in either a different clock time of administration but at the correct circadian time for that individual.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to systems and methods for timing the administration of therapies according to an individual's circadian rhythm. A first embodiment of the invention is directed to a method of determining and administering therapies responsive to a circadian rhythm of a user comprising receiving an indication of a therapy to be administered to the user, identifying one or more periods during a circadian cycle during which administration of the therapy is preferred, defining a circadian administration time, receiving an indication of a normal circadian rhythm of the user, determining the position of the circadian administration time within the normal circadian rhythm of the user, defining a user circadian administration time, determining a time of day corresponding to the user circadian administration time, defining a clock administration time, and at least one of providing an indication to the user to administer the therapy at the clock administration time and administering the therapy to the user at the clock administration time.

In some embodiments, providing an indication to the user to administer the therapy may comprise at least one of illuminating an indicator light, operating a sound-generating device to provide an audio indication, operating a visual display to display a message, and transmitting a message to be received on a computerized device. In some embodiments, administering the therapy to the user at the clock administration time may comprise at least one of dispensing a medication associated with the therapy such that the medication may be retrieved for administration and operating a machine to administer the therapy.

In some embodiments, the method may further comprise receiving an indication of when the therapy was administered, defining an administration time, determining an estimated circadian phase coinciding with the administration time, determining an estimated efficacy of the therapy responsive to the estimated circadian phase, determining a second user circadian administration time to administer a second administration of the therapy responsive to at least one of the estimated efficacy, the circadian administration time, and the user circadian administration time, determining a second clock administration time, and at least one of providing an indication to the user to administer the second administration of the therapy at the second clock administration time and administering the second administration of the therapy to the user at the second clock administration time.

In some embodiments the normal circadian rhythm of the user may be at least one of a central circadian clock of the user and a peripheral circadian clock of the user.

In some embodiments, the method may further comprise receiving an indication of an expected transition of the circadian rhythm of the user, identifying one or more periods during a circadian cycle during which administration of the therapy is preferred to facilitate the transition of the circadian rhythm of the user, defining a transition circadian administration time, determining the position of the transition circadian administration time within the normal circadian rhythm of the user, defining a user transition circadian administration time, determining a time of day corresponding to the user transition circadian administration time responsive to the normal circadian rhythm of the user, defining a transition clock administration time, and at least one of providing an indication to the user to administer the therapy at the transition clock administration time and administering the therapy to the user at the transition clock administration time.

In some embodiments, the method may further comprise receiving an indication of a present circadian phase of the user and the clock administration time may be determined responsive to the present circadian phase of the user. In further embodiments, the present circadian phase may be a first present circadian phase and the method may further comprise receiving an indication of when the therapy was administered, defining an administration time, determining an estimated circadian phase coinciding with the administration time, determining an estimated efficacy of the therapy responsive to the estimated circadian phase, determining a second user circadian administration time to administer a second administration of the therapy responsive to at least one of the estimated efficacy, the circadian administration time, the user circadian administration time, and a second indication of the present circadian phase of the user, determining a second clock administration time responsive to the second user circadian administration time, and at least one of providing an indication to the user to administer the second administration of the therapy at the second clock administration time and administering the second administration of the therapy to the user at the second clock administration time. Other embodiments The method of claim 7 wherein the indication of the present circadian phase of the user and the normal circadian rhythm of the user is at least one of the user's sleep-wake cycles, light-dark exposure, chronotype, sex, age, present time zone, future time zone, present work shift, future work shift, heat rate, heart rate variability, core temperature, skin temperature, and a biological marker.

In some embodiments, the therapy is a first therapy, the circadian administration time is a first therapy circadian administration time, the user circadian administration time is a first therapy user circadian administration time, the clock administration time is a first therapy clock administration time, and the indication provided to the user to administer the therapy is a first indication. The method may further comprise receiving an indication of a second therapy to be administered, identifying one or more periods during a circadian cycle during which administration of the second therapy is preferred responsive to the first therapy also being administered, defining a second therapy circadian administration time, determining the position of the second circadian administration time within the normal circadian rhythm of the user, defining a second therapy user circadian administration time, determining a time of day corresponding to the second therapy user circadian administration time, defining a second therapy clock administration time, and at least one of providing a second indication to the user to administer the second therapy at the second therapy clock administration time and administering the second therapy to the user at the second therapy clock administration time. The first therapy circadian administration time may be determined responsive to the second therapy also being administered.

In further embodiments, the method may further comprise receiving an indication of a present circadian phase of the user. Each of the first clock administration time and the second clock administration time may be determined responsive to the present circadian phase of the user. In other further embodiments, providing the first indication to the user to administer the first therapy may comprise at least one of illuminating a first indicator light, operating a sound-generating device to provide a first audio indication, operating a visual display to display a first message, and transmitting a first message to be received on a computerized device. Providing the second indication to the user to administer the first therapy may comprise at least one of illuminating a second indicator light that is differentiated from the first indicator light, operating a sound-generating device to provide a second audio indication that is differentiated from the second audio indication, operating a visual display to display a second message that is differentiated from the first message, and transmitting a second message that is differentiated from the first message to be received on the computerized device.

In other further embodiments, administering the first therapy to the user may comprise at least one of operating a first machine to enable a first medication associated with the first therapy to be retrieved for administration and operating a first machine to administer the first therapy, and administering the second therapy to the user comprises at least one of operating one of the first machine and a second machine to enable a second medication associated with the second therapy to be retrieved for administration, the second medication being differentiated from the first medication, and operating at least one of the first machine and a second machine to administer the second therapy that is differentiated from the first therapy.

Another embodiment of the invention is directed to a system for determining and facilitating administration of therapies responsive to a circadian rhythm of a patient comprising a network communication device configured to receive an indication of a therapy to be administered to the patient, receiving an indication of a normal circadian rhythm of the patient, and transmit a signal to a remote computerized device configured to at least one of provide an indication to administer the therapy at a clock administration time and administer the therapy at the clock administration time. The system may further comprise a processor positioned in communication with the network communication device and configured to identify one or more periods during a circadian cycle during which administration of the therapy is preferred, defining a circadian administration time, determine the position of the circadian administration time within the normal circadian rhythm of the user, defining a user circadian administration time, and determine a time of day corresponding to the user circadian administration time, defining a clock administration time. The system may further comprise a non-transitory storage medium positioned in communication with each of the processor and the network communication device.

### BRIEF DESCRIPTION OF THE DRAWINGS

Some embodiments of the present invention are illustrated as an example and are not limited by the figures of the accompanying drawings, in which like references may indicate similar elements.

FIG. 1 is a flow chart illustrating a method according to an embodiment of the invention.

FIG. 2 is a flow chart illustrating another method according to an embodiment of the invention.

FIGS. 3A-B are flow charts illustrating another method according to an embodiment of the invention.

FIGS. 4A-B are flow charts illustrating another method according to an embodiment of the invention.

FIGS. 5A-B are flow charts illustrating another method according to an embodiment of the invention.

FIG. 6 is a schematic view of a system and connected devices according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides methods and a system for timing the administration of therapies responsive to an individual's circadian rhythm. These methods may be performed by a computerized device having the necessary hardware configured to perform the steps of the methods as described below.

The terms “user,” “individual,” and “patient” may be used throughout the specification. Except where specifically identified as a separate individual (e.g., the user of the method is administering therapy to a patient whose circadian rhythm is being monitored), these terms may be used interchangeably and

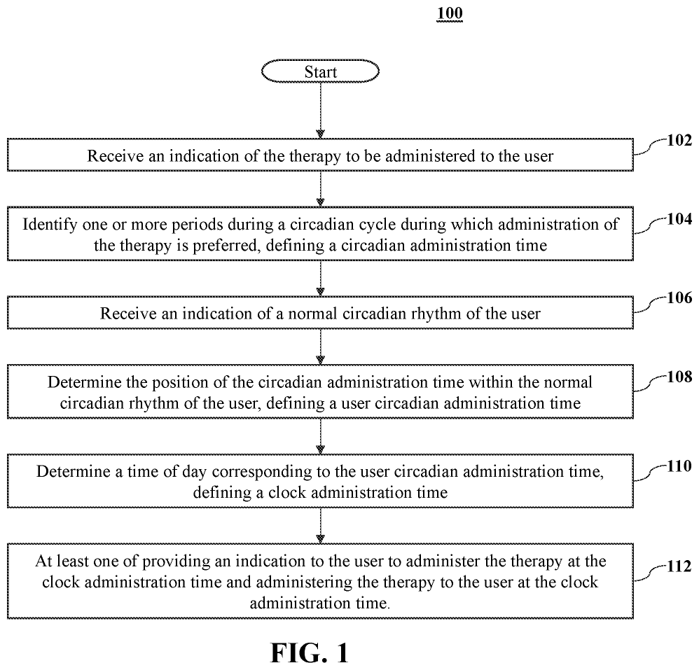

Referring specifically to FIG. 1, a method 100 of determining and administering therapies responsive to a circadian rhythm of a patient is presented. The method may include receiving an indication of the therapy to be administered at 102. As noted above, the therapy may be any type of therapy intended to have a therapeutic effect, including, but not limited to, the administration of medication, vaccines, and other pharmaceutical products, and other therapies such as chemotherapy and surgery.

Upon receiving the indication of the therapy to be administered, the method 100 may continue at 104 with identifying one or more periods during a circadian cycle during which administration of the therapy is preferred. The identified periods may be defined as a circadian administration time. Such identification may be accomplished by querying a database of therapies with the received therapy to be administered and receiving a response indicating the period during the circadian cycle which that particular therapy is most efficacious. Examples of such periods may include one or more phases during the circadian rhythm, such as sleep phase and/or wake phase, as well as positions within different phases, such as early, middle, or late within the respective phases. These positions within the circadian rhythm are exemplary only and all possible characterizations of the phases of the rhythm are contemplated and included within the scope of the invention.

The method 100 may at 106 continue with receiving an indication of a normal circadian rhythm of the user. Such a normal circadian rhythm may conform to one of the standard types of rhythms identified, including a rhythm that conforms to a typical sleep-wake cycle, so-called “night owl” rhythms with later wake-up and going to sleep times compared to a typical sleep-wake cycle, so-called “early bird” rhythms with earlier wake-up and going to sleep times compared to a typical sleep-wake cycle, cycles with comparatively shorter or longer sleep phases, cycles with comparatively shorter or longer wake phases, cycles with sporadic or inconsistent sleep-wake cycles, and combinations thereof, commonly referred to as chronotype. This indication may include an indication of when the user wakes up, when the user goes to sleep, when the user is typically awake, when the user is typically a sleep, whether the user is a night owl, an early bird, or has a typical circadian pattern, their light-dark exposure, and the like. Additional indications may be the user's sex, age, present time zone, future time zone, present work shift, future work shift, heart rate, heart rate variability, core body temperature, skin temperature, and any other biological marker as is known in the art.

As is known in the art, there are several circadian clocks for an individual. The central circadian rhythm is governed by the central suprachiasmatic nucleus (SCN) of the hypothalmus, but there are several so-called peripheral circadian clocks that exist in various other tissues in an individual and are synchronized by the SCN, including, but not limited to, the liver, kidney, lung, fibroblasts, glands, including submandibular glands, and the like. It is contemplated and included within the scope of the invention that the normal circadian rhythm of the user may be at least one of the central circadian rhythm and a peripheral circadian rhythm.

The method 100 may continue at 108 with determining the position of the circadian administration time within the normal circadian rhythm of the user, defining a user circadian administration time. Where the user has a standard circadian rhythm, with typical sleep-wake cycles in terms of length and time of day, there may be no difference in the circadian administration time and the normal circadian rhythm, i.e. the circadian administration time may occur at a typical time within the normal circadian rhythm. Where the normal circadian rhythm of the user deviates from a typical circadian rhythm, the position of the circadian administration time may similarly deviate from its position within a normal circadian rhythm. For example, it may be possible that the circadian administration time is relative to a hormonal change in the user, for example, the therapy is most efficacious when administered one hour after a hormonal increase. Furthermore, a user's normal circadian rhythm may be abnormal in that the hormonal increase occurs at an unusual time within their circadian rhythm. Accordingly, the circadian administration time may be different for that user when compared to an individual with a typical circadian rhythm. Other physiological changes tied to circadian rhythms are contemplated and included within the scope of the invention, including, but not limited to, blood glucose level, body temperature, and the like.

The method 100 may continue at 110 with determining a time of day corresponding to the user circadian administration time, defining a clock administration time. This step may produce a time during they day during which the therapy may be administered for maximum efficacy. Accordingly, the clock administration time is the time shown on the clock when the therapy may be administered, e.g. 9:00 a.m., 13:00, etc. Crucially, the clock administration time is determined responsive to the user circadian administration time which is itself responsive to the normal circadian rhythm of the user. Accordingly, user's receiving the same therapy that have different normal circadian rhythms may have different clock administration times, as the maximum efficacy period of their respective circadian rhythms occur at different points in time during the day.

Finally, the method 100 may conclude at 112 with performing an additional action that may take many forms, including providing an indication to the user to administer the therapy at the clock administration time and administering the therapy to the user at the clock administration time. The notification may take many forms, including all possible methods of conveying information to the user. In some embodiments, this may include operating a device that provides audio, visual, and/or combinations thereof to convey the information. In some embodiments, this may include operating a display device to provide text or illustration that conveys the clock administration time for the therapy, for example, displaying the therapy to be provided and the clock administration time for the therapy. In some embodiments, this may include operating a sound-generating device to provide an audible indication of the clock administration time that may include announcing the time, announcing the therapy to administer, and a tonal indication that is associated with the therapy/clock administration time. In some embodiments, the indication may include transmitting a message to a remote computerized device to provide an indication on the remote computerized device indicating the clock administration time. This may include one or more of sending a command to an application running on the remote computerized device, such as a push notification, sending a text message complying with a known text messaging protocol, for example, short message service (SMS), multimedia messaging service (MMS), enhanced messaging service (EMS), rich communication service (RCS), and the like. This may further include sending an e-mail to an e-mail address associated with the user. This may further include interacting with a remote computerized device operable to provide an audio indication as described above, such as a smart home device, including, but not limited to, Amazon Echo devices, Google Home devices, and Apple Homepod devices.

It is further contemplated and included within the scope of the invention that multiple indications may be provided to the user regarding administration of the therapy at the clock administration time. For example, one or more indications may be provided to the user in advance of the clock administration time so that the user has time to prepare for therapy administration, either separate from or in addition to an indication occurring at the clock administration time.

In some embodiments, administering the therapy may include operating one or more devices configured to facilitate the administration of the therapy. This may include any device that either facilitates the administration of therapy and/or administers therapy. Some embodiments may include operating a device that provides access to medication, vaccines, or other pharmaceuticals materials. Such access may include dispensing the materials from a secured location that is inaccessible to a location that is accessible, unlocking or opening a container containing the materials so that they become accessible, and the like. Some embodiments may include administering the therapy, including, for example, operating a material-dispensing machine such as a medication-dispensing pump to inject the medication into the patient, operating a machine that performs the therapy, for example performs physical therapy, irradiates the patient with therapeutic radiation, and the like, and all other therapy-delivery devices as are known in the art.

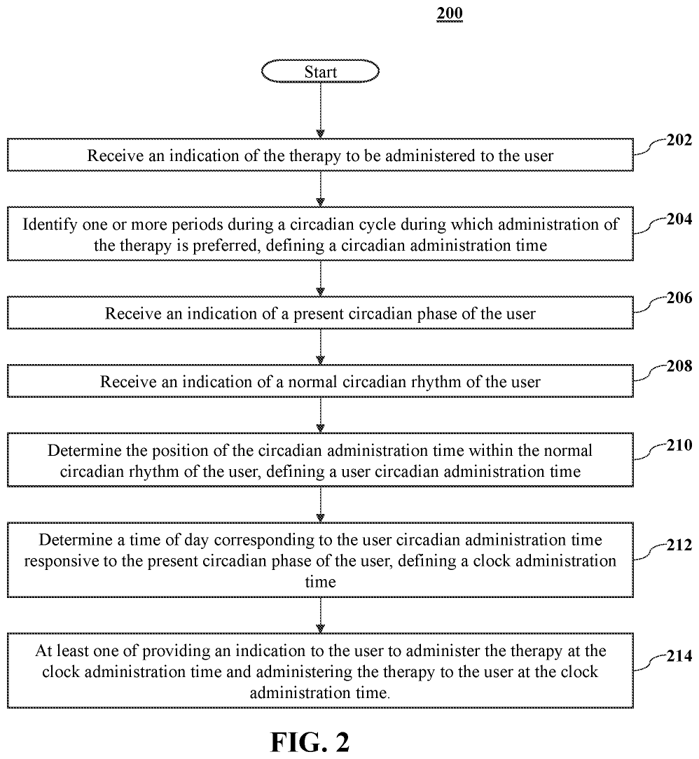

Referring now to FIG. 2, a method 200 according to another embodiment of the invention is presented. The method 200 may include steps similar to those of the method 100 of FIG. 1, including receiving an indication of the therapy to be administered to the user at 202 and identifying one or more periods during a typical circadian cycle during which administration of the therapy is preferred, defining a circadian administration time at 204. The method 200 may continue at 206 with receiving an indication of a present circadian phase of the user. This may indicate at what point in their circadian rhythm the user is presently in. Such an indication may include a physical or physiological measurement, including, but not limited to, body temperature, such as a minimum core body temperature, measured hormone levels such as melatonin, cortisol, and/or adrenocorticotropic hormone (ACTH), blood glucose level, and the like. Such an indication may also include when and how long the user has been awake, when and how long the user most recently slept, when and how much circadian cycle-affecting substances the user has ingested (such as caffeine, melatonin supplements, and the like), when and the extent of recent exercise of the user, and the like, and when and what the user has most recently eaten for nutrition. These examples are for illustrative purposes only and do not limit the scope of types of indications contemplated by the invention.

The method 200 may continue at 208 with receiving an indication of a normal circadian rhythm of the user, as described above, and at 210 with determining the position of the circadian administration time within the normal circadian rhythm of the user, defining a user circadian administration time. The method 200 may continue at 212 with determining a time of day corresponding to the user circadian administration time, defining a clock administration time. In this embodiment, the clock administration time is determined responsive to the present circadian phase of the user. This may facilitate determining a clock administration time that is more accurate based on the circadian rhythm of the user for that day, instead of relying only on the normal circadian rhythm for the user. Accordingly, this results in the therapy being administered as closely as possible to the circadian administration time as possible while accounting for the day-to-day variations in the circadian rhythm of individuals.

The method 200 may conclude at 214 with at least one of providing an indication to the user to administer the therapy at the clock administration time and administering the therapy to the user at the clock administration time.

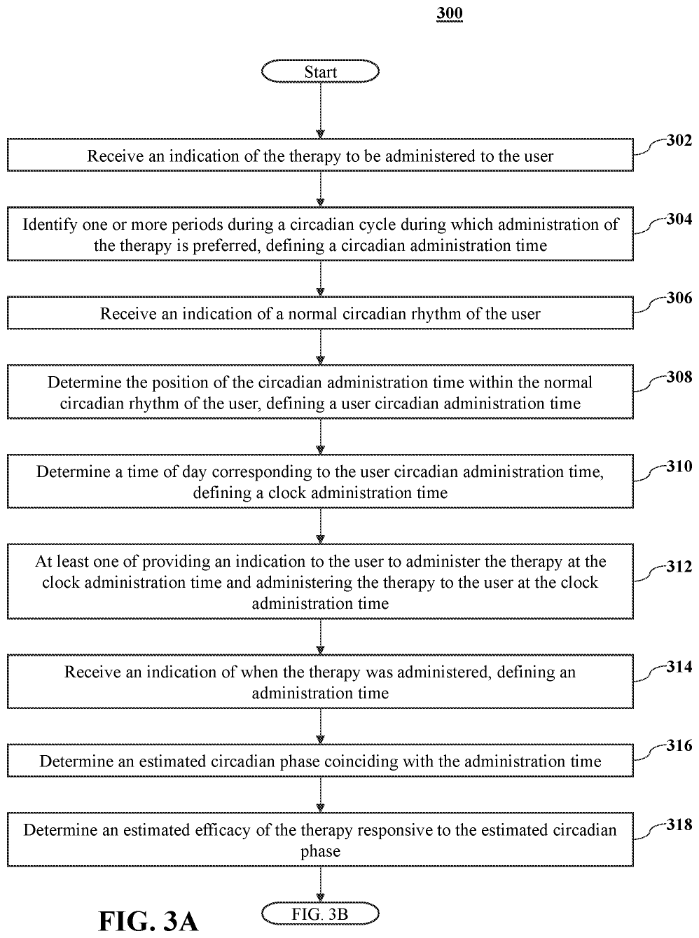

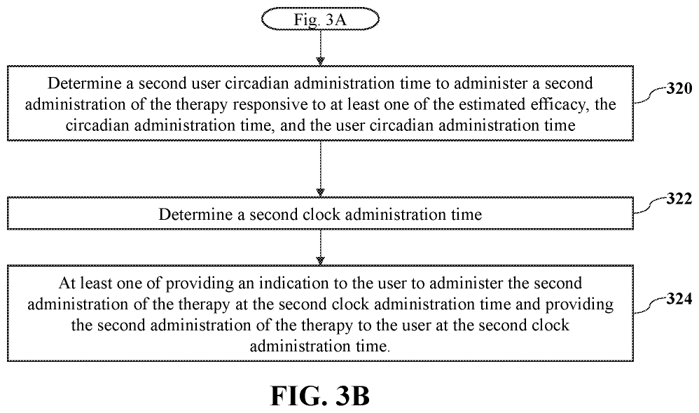

Referring now to FIGS. 3A-B, a method 300 according to another embodiment of the invention is presented. The method 300 may include many of the steps of the method 100 of FIG. 1, including receiving an indication of the therapy to be administered to the user at 302, identifying one or more periods during a typical circadian cycle during which administration of the therapy is preferred, defining a circadian administration time at 304, and receiving an indication of a normal circadian rhythm of the user at 306. The method 300 may then continue at 308 with determining a time of day corresponding to the user circadian administration time, defining a clock administration time, and at 312 with at least one of providing an indication to the user to administer the therapy at the clock administration time and administering the therapy to the user at the clock administration time.

The method 300 may continue at 314 with receiving an indication when the therapy was administered, defining an administration time. The indication may include various types of information, including, but not limited to, and indication when the therapy was performed, when the therapy started, when the therapy was completed, the duration of the therapy, and a quantification of some aspect of the therapy, such as a dosage of medication administered, a dosage of radiation administered, a length of time of performing a type of physical therapy or therapeutic exercise, and the like, and a qualification of some aspect of the therapy, such as the relative quality (e.g. excellent, good, fair, poor) of the therapy that was administered.

The method 300 may continue at 316 with determining an estimated circadian phase coinciding with the administration time. This estimation may be made responsive to the administration time and the normal circadian rhythm received at 306. As described above, the efficacy or other desirable trait of a therapy can vary significantly by when in the circadian rhythm it is administered. Accordingly, after determining the estimated circadian phase coinciding with the administration time, the method 300 may continue at 318 with determining an estimated efficacy of the therapy responsive to the estimated circadian phase. The estimated efficacy may be determined responsive to the administration time. Moreover, the database of therapies described above may be queried if it is determined the administration time is outside one or both of the circadian administration time and the user circadian administration time. In such embodiments, the database may have information about the efficacy of therapies when administered in circadian phases other than the preferable circadian phase.

The method 300 may continue at 320 with determining a second user circadian administration time to administer a second administration of the therapy responsive to at least one of the estimated efficacy, the circadian administration time, and the user circadian administration time. The circadian timing of the second user circadian administration time may be calibrated to compensate for the less-than-completely efficacious first administration of the therapy. For example, the second administration of the therapy may be necessary because of the estimated efficacy being under a threshold efficacy. The threshold efficacy may be a minimum efficacy necessary for adequate therapeutic effect of the therapy. Accordingly, in some embodiments the second user circadian administration time may be selected for a period during the user's circadian rhythm that is not maximally efficacious, so that there is not too much of the effect caused by the therapy than is desirable. In some embodiments, the second user circadian administration time may be the next in time most efficacious period during the user's circadian rhythm within the same day to have sufficient therapeutic effect within the single circadian cycle. Alternatively, the second administration of the therapy may be part of a prescribed course of treatment, but may nonetheless be selected based on the efficacy of the first administration of the therapy. Furthermore, it is contemplated and included within the scope of the invention that modifications to the therapy, i.e. differences between the first and second administrations of the therapy may also be determined. Examples of such modifications include increasing or reducing the amount of therapeutic substance that is delivered to the patient, changing the length or intensity of administration of a therapy, and the like.

The method 300 may continue at 322 with determining a second clock administration time responsive to the second user circadian administration time and at 324 with at least one of providing an indication to the user to administer the second administration of the therapy at the second clock administration time and providing the second administration of the therapy to the user at the second clock administration time.

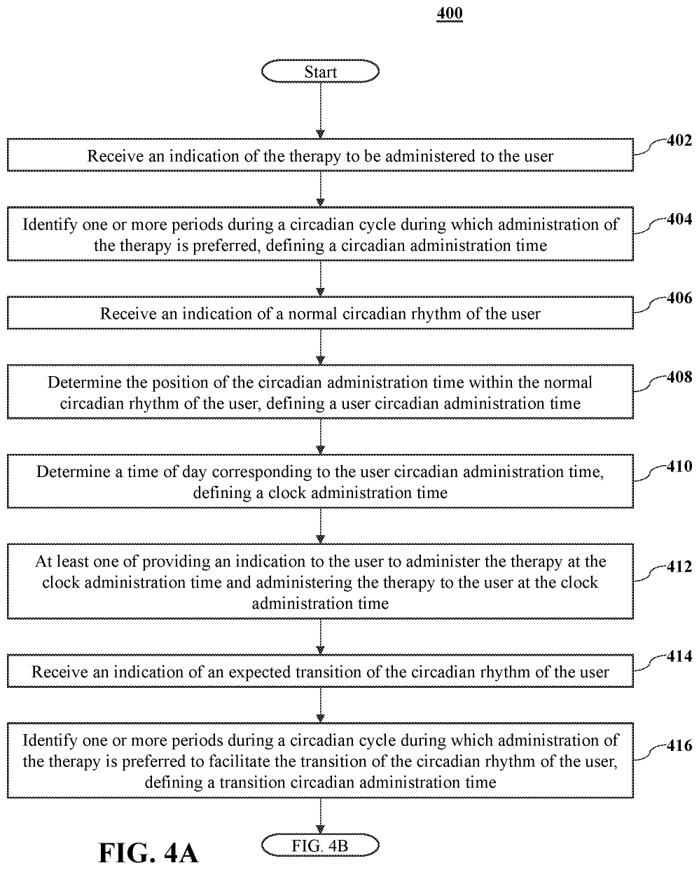

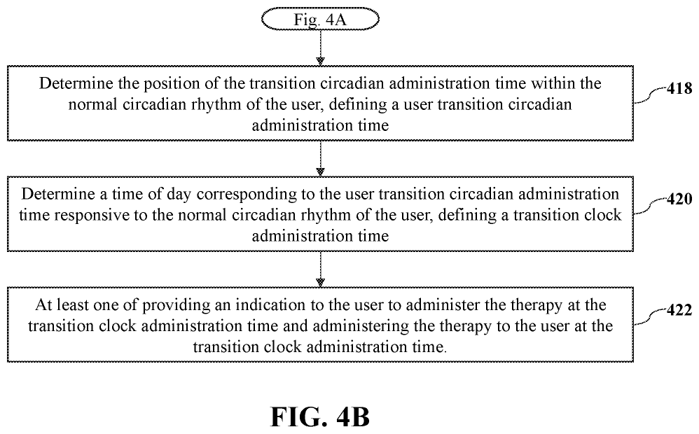

Referring now to FIGS. 4A-B, a method 400 according to an embodiment of the invention is presented. The method 100 may be similar to method 100 of FIG. 1, including receiving an indication of a therapy to be administered to the user at 402, identifying one or more period during a circadian cycle during which administration of the therapy is preferred, defining a circadian administration time at 404, receiving an indication of a normal circadian rhythm of the user at 406, determining the position of the circadian administration time within the normal circadian rhythm of the user, defining a user circadian administration time, at 408, determining a time of day corresponding to the user circadian administration time, defining a clock administration time, at 410, and at least one of providing an indication to administer the therapy at the clock administration time and administering the therapy to the user at the clock administration time at 410. The method 400 may continue at 414 with receiving an indication of an expected transition of the circadian rhythm of the user. The expected transition of the circadian rhythm may be any event or cause that may effectuate a change in the circadian rhythm of the user, including, but not limited to, upcoming travel or move to a different time zone for the user, an upcoming change to the sleep schedule for the user for whatever reason, including a change in the work schedule (e.g. changing to a night shift where the user works during the night hours and sleeps during the day), an anticipated change to the circadian rhythm originating with starting or terminating an activity or therapy that changes the user's circadian rhythm (e.g. starting or stopping taking medication that changes the sleep habits of the user), and the like. The indication of the expected transition may indicate both the direction of the circadian shift (e.g. rising earlier or later) and/or the magnitude of the circadian shift (e.g. how much earlier/later rising occurs).

The method 400 may continue at 416 with identifying one or more periods during a circadian cycle during which administration of the therapy is preferred to facilitate the transition of the circadian rhythm of the user, defining a transition circadian administration time. Accordingly, any effect administration of the therapy may have may be identified and considered to determine when to administer the therapy so as to assist the user in transitioning to the new circadian rhythm. In some instances, where the therapy has no circadian effect, the transition circadian administration time may be the same as the circadian administration time. However, where the therapy has a circadian effect, that effect may be utilized to help the user shift to the new circadian rhythm. Additional information regarding identifying when to administer therapies to effectuate a circadian shift may be found in U.S. patent application Ser. No. 16/432,544 titled Method and System for Generating and Providing Notifications for a Circadian Shift Protocol filed Jun. 5, 2019, the content of which is incorporated by reference herein in its entirety except to the extent disclosure therein is inconsistent with disclosure herein. The information about the circadian effect the therapy will have may be obtained similar to identifying the preferred circadian point/phase to administer the therapy, by querying a database containing such information and receiving an indication as to the circadian effect of the therapy.

The method 400 may continue at 418 with determining the position of the transition circadian administration time within the normal circadian rhythm of the user, defining a user transition circadian administration time. This may be performed responsive to the indicated circadian shift the therapy will affect. For example, if the therapy will have an advance effect and the transition is an advance, the user transition circadian administration time may be at the same time or earlier than the user circadian administration time. As another example, if the therapy will have a delay effect and the transition is a delay, the user transition circadian administration time may be at the same time or later than the user circadian administration time. These scenarios are exemplary only and any combination of advances and delays and user transition circadian administration times responsive thereto are contemplated and included within the scope of the invention.

The method 400 may continue at 420 with determining a time of day corresponding to the user transition circadian administration time responsive to the normal circadian rhythm of the user, defining a transition clock administration time, and at 420 with at least one of providing an instruction to the user to administer the therapy and administering the therapy to the user at the transition clock administration time.

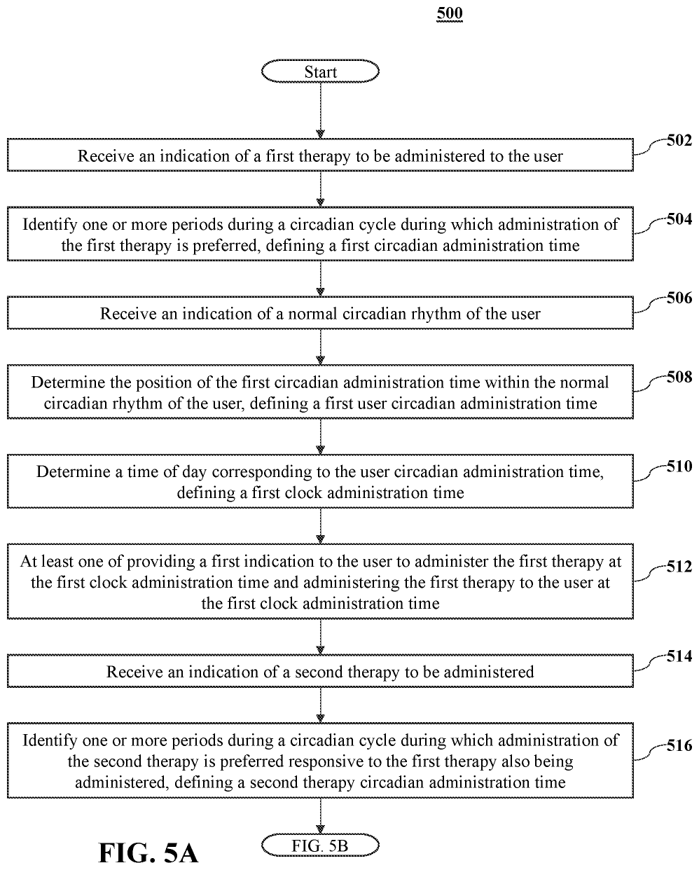

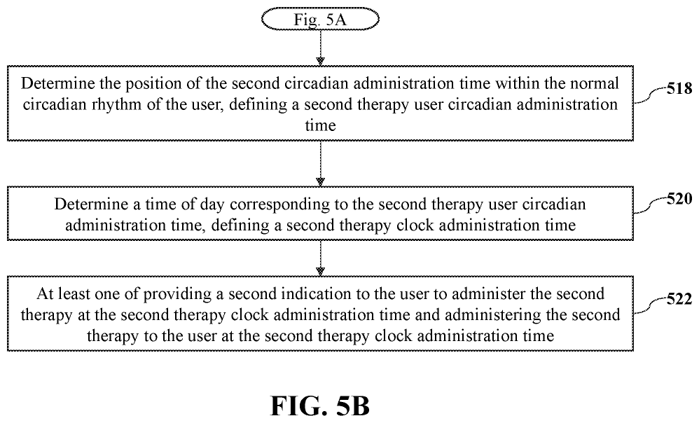

Referring now to FIGS. 5A-B, a method 500 according to another embodiment of the invention is presented. The method 500 may include receiving an indication of a first therapy to be administered at 502, identifying one or more periods during a circadian cycle during which administration of the first therapy is preferred, defining a first circadian administration time, at 504, and receiving an indication of a normal circadian rhythm of the user at 506. The method 500 may further continue at 508 with determining the position of the first circadian administration time within the normal circadian rhythm of the user, defining a first user circadian administration time, at 510 with determining a time of day corresponding to the user circadian administration time, defining a first clock administration time, and at 512 with at least one of providing a first indication to the user to administer the first therapy at the first clock administration time and administering the first therapy to the user at the first clock administration time.

The method 500 may further continue at 514 with receiving a second therapy to be administered. The second therapy may be completely distinct from the first therapy, e.g. is prescribed as a result of a separate diagnosis, or it may be related to the first therapy, e.g. the first and second therapies are prescribed together as a treatment regimen. This embodiment may be distinguished from the method 300 of FIGS. 3A-B where a second administration of the same treatment (in this embodiment, the first treatment) is administered. The method 500 may continue at 516 with identifying one or more periods during the circadian cycle during which administration of the second therapy is preferred, defining a second therapy circadian administration time. In the present embodiment, this identification may be made responsive to that the first therapy is also being administered. In this way, any interaction or interference arising from administration of the first therapy may be accounted for and minimized, maximized, or avoided depending on the desired effect of the interaction. In some embodiments, the second therapy circadian administration time may be determined without consideration of the administration of the first therapy. In some embodiments, where a contraindication/effect of the first therapy is sufficiently great that it may substantially negatively effect the efficacy of the second therapy at the preferred and/or any point in the circadian rhythm, a warning may be provided to the user by an auditory or visual means as described for providing an indication to the user as described above.

The method 500 may continue at 518 with determining the position of the second administration time within the normal circadian rhythm of the user, defining a second therapy user circadian administration time, at 520 with determining a time of day corresponding to the second therapy user circadian administration time, defining a second therapy clock administration time, and at 522 with at least one of providing a second indication to the user to administer the second therapy at the second therapy clock administration time and administering the second therapy to the user at the second therapy clock administration time. The second indication may be of the same or similar type of indication as the first indication or it may be completely different. In some embodiments, the second indication may be distinguishable from the first indication so that an observer, such as the user, may perceive the first indication and understand it is related to the administration of the first therapy and the second indication and understand it is related to the administration of the second therapy. For example, a first lighting device configured to emit light within a first wavelength range that is perceived by an observer as a first color may indicate the first therapy and a second lighting device configured to emit light within a second wavelength range that is perceived by an observer as a second color may indicate the second therapy.

Additionally, administration of the first and second therapies may take the form of alternatives of the methods of administering therapy as described above. For example, a first IV pump may be operated to administer the first therapy and a second IV pump may be operated to administer the second therapy. As another example, a pill box containing a plurality of compartments may be operated to open a first compartment thereof to permit access to a first medication associated with the first therapy and further operated to open a second compartment thereof to permit access to a second medication associated with the second therapy. As another example, an IV pump may be operated to administer the first therapy and a pill box may be operated to open a container thereof to facilitate access to a medication associated with the second therapy. In this way, operation of any number of devices to administer or facilitate administration of the first and second therapies is contemplated and included within the scope of the invention.

Furthermore, it is contemplated that an indication of the first clock administration time may be provided and operation of a device to administer or facilitate administration of the second therapy within the method, and vice-versa, i.e. operating a device to administer or facilitate administration of the first therapy and providing an indication of the second clock administration time.

While two therapies are disclosed, it is contemplated that the method 500 may include any number of therapies, and each subsequent therapy may be identified, and a clock administration time determined therefore, responsive or not responsive to the clock administration times of the preceding therapies.

FIGS. 1-5 depict methods with varying functionality. It is contemplated and included within the scope of the invention that any of the functions that differ between the embodiments depicted in those figures may be selectively combined in all possible permutations, such that a method may include any and all elements of the methods shown in FIGS. 1-5 and their attending description. All permutations of such combinations are contemplated and included within the scope of the invention.

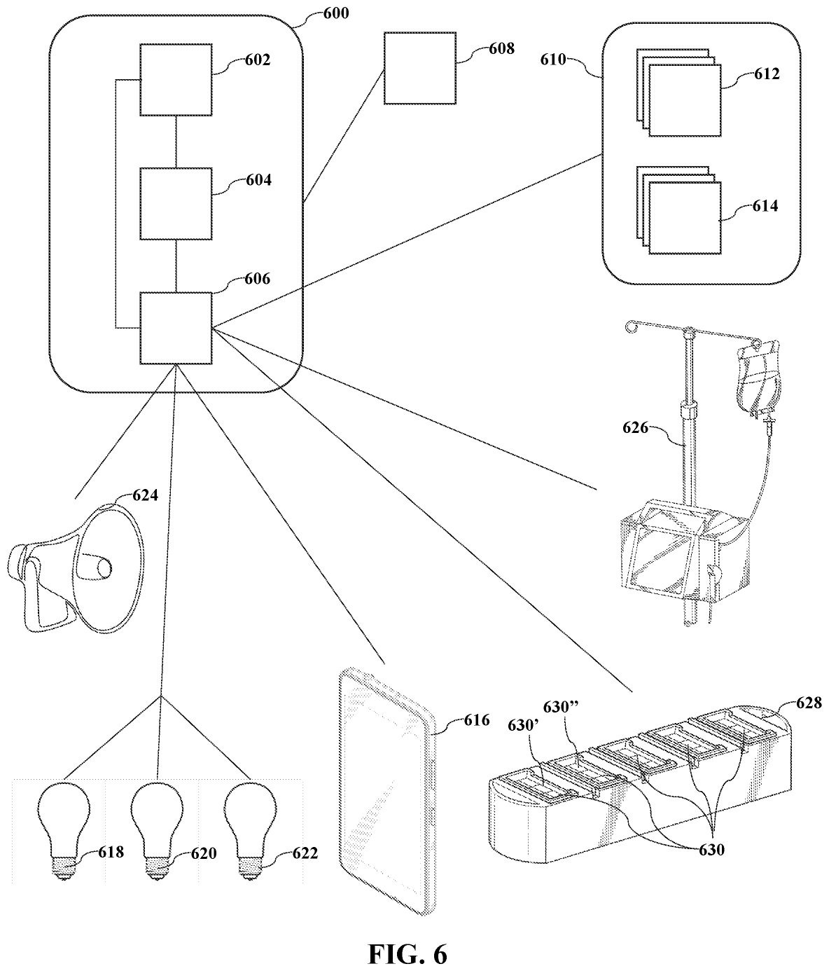

Referring now to FIG. 6, a system 600 capable of performing the above-described methods is presented. The system 600 may be a computerized device operable to receiving the indications of the treatment, the user's normal circadian rhythm, and indications of the user's present position in their circadian rhythm, as well as indications about upcoming changes to the circadian rhythm, as described above. The system 600 may include a processor 602. The processor 602 may be any type of processing device as is known in the art, including, but not limited to, microprocessors. Integrated circuits (ICs), field-programmable gate arrays (FPGAs), and the like. The system 600 may further comprise a memory device 604. The memory device 604 may be in communication with the processor 602 and operable to store data and software thereon. The memory device 604 may be a non-transitory storage medium. The memory device 604 may be any type of storage device as is known in the art, including, but not limited to, a hard disk or other physical storage medium, a solid state drive (SSD) or any other device that uses flash memory or other electronic non-volatile computer memory storage medium, and all other storage medium devices, including non-volatile storage mediums. The system 600 may further comprise a network communication device 606 positioned in communication with one or both of the processor 602 and the memory device 604. The network communication device 606 may be configured to send and receive data to remote computerized devices using any communication medium, including wired and wireless (such as radio and light-based), and communication protocol as is known in the art, including, but not limited to, Ethernet, Universal Serial Bus (USB), 802.XX protocols including Wi-Fi, Bluetooth, Zigbee, Z-Wave, LiFi, cellular communication including CDMA, TDMA, 3G, 4G, and 5G, as well as messaging protocols such as SMS, MMS, and RCS. Moreover, the network communication device may be operable to communicate any type of wide area network (WAN) as is known in the art, including the Internet and cellular communication networks, local area network (LAN) such as an Ethernet or WiFi network, and personal area network (PAN) such as a Bluetooth network. Furthermore, the system 600 may comprise a plurality of network communication devices 606 to facilitate communication across one or more of these mediums and/or protocols. Additionally, the system 600 may comprise software to facilitate communication across one or more of these mediums utilizing third-party services, e.g. sending an SMS message utilizing third-party services across the Internet.

In some embodiments, the system 600 may further comprise a user input device 608. The user input device 608 may be any device capable of receiving an input from a user, including, but not limited to, a mouse, a keyboard, a touchscreen, a microphone, a camera, pushbuttons, switches, toggles, trackpads, and the like.

The system 600 may be positioned in communication with one or more remote computerized devices, specifically a server 610 that comprises one or more database 612, 614 the contain information regarding therapies and their relationship to circadian rhythms as described above. It is contemplated and included within the scope of the invention that the system 600 may communicate with multiple servers or other devices that comprises databases to obtain all necessary information for performing the above-described methods.

Additionally, the system 600 may be positioned in communication with one or more indicating devices. Types of indicating devices include remote computerized devices, such as a smartphone 616, one or more light-emitting devices 618, 620, 622, and an audio device 624 such as a speaker. The system 600 may communicate with these devices utilizing the network communication device 606 or other computer hardware as is known in the art, including an audio output device, such as a sound card. The system 600 may send messages to the smartphone 616 by any means or method as is known in the art, including push notifications to software running on the smartphone 616, SMS, MMS, or RCS messages, or placing an automated telephone call that the user may receive and hear an automated announcement regarding administering a therapy. Additionally, as described above, the system may generate either a tonal indication, a spoken indication, or other audible indication using the speaker 624. Furthermore, the system 600 may control the operation of one or more of the light-emitting devices 618, 620, 622 to indicate administration of a therapy. As suggested above, the light emitting devices 618, 620, 622 may each be configured to emit light within a different wavelength range that is perceived by an observer as different colors, and those colors may be associated with different therapies, and the system may selectively illuminate one or more to indicate the different therapies are to be administered. In some embodiments, a lighting device may be operable to selectively emit light within different wavelength ranges perceived as different colors, the different colors being associated with different therapies, and the system may be configured to selectively illuminate the lighting device to emit the different colors in indicate the therapies to be administered.

Additionally, the system 600 may be configured to operate one or more devices configured to administer therapies or facilitate the administration of therapies. For example, the system 600 may be positioned in operative communication with an IV pump 626 operable to dispense medication into a patient. The system 600 may be configured to operate the IV pump 626 to administer a therapy as described above. As another example, the system 600 may be positioned in operative communication with a pill box 628. The pill box 628 may comprise a plurality of lidded compartments 630 that may be operated by the system 600 to selectively open. The system 600 may be configured to selectively operate the pill box 628 to open a first compartment 630′ of the plurality of compartments 630 comprising a first medication associated with a first therapy as described above, to open a second compartment 630″ of the plurality of compartments 630 comprising a second medication associated with a second therapy as described above, etc., thereby facilitating retrieval of the medications and administration thereof. These devices are exemplary only; the system 600 may be positioned in operative communication with any device that may administer or facilitate administration of therapy as is known in the art and configured to selectively operate the device to administer or facilitate administration therapy as described above. Moreover, the system 600 may be positioned in communication with multiple devices of varying operation and capabilities, e.g. an IV pump 626 and a pill box 628 as described herein, and configured to selectively operate those devices to administer or facilitate administration of therapy. Furthermore, it is contemplate and included within the scope of the invention that one or more of the processor 602, the network communication device 606, or other hardware as may be comprised by the system 600 may enable it to operate the devices.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. A method of determining and administering therapies responsive to a circadian rhythm of a user comprising:
receiving an indication of a therapy to be administered to the user;
identifying one or more periods during a circadian cycle during which administration of the therapy is preferred, defining a circadian administration time;
receiving an indication of a normal circadian rhythm of the user;
determining the position of the circadian administration time within the normal circadian rhythm of the user, defining a user circadian administration time;
determining a time of day corresponding to the user circadian administration time, defining a clock administration time; and
at least one of providing an indication to the user to administer the therapy at the clock administration time and administering the therapy to the user at the clock administration time.

2. The method of claim 1 wherein providing an indication to the user to administer the therapy comprises at least one of illuminating an indicator light, operating a sound-generating device to provide an audio indication, operating a visual display to display a message, and transmitting a message to be received on a computerized device.

3. The method of claim 1 wherein administering the therapy to the user at the clock administration time comprises at least one of dispensing a medication associated with the therapy such that the medication may be retrieved for administration and operating a machine to administer the therapy.

4. The method of claim 1 further comprising:
receiving an indication of when the therapy was administered, defining an administration time;
determining an estimated circadian phase coinciding with the administration time;
determining an estimated efficacy of the therapy responsive to the estimated circadian phase;
determining a second user circadian administration time to administer a second administration of the therapy responsive to at least one of the estimated efficacy, the circadian administration time, and the user circadian administration time;
determining a second clock administration time; and
at least one of providing an indication to the user to administer the second administration of the therapy at the second clock administration time and administering the second administration of the therapy to the user at the second clock administration time.

5. The method of claim 1 wherein the normal circadian rhythm of the user is at least one of a central circadian clock of the user and a peripheral circadian clock of the user.

6. The method of claim 1 further comprising:
receiving an indication of an expected transition of the circadian rhythm of the user;
identifying one or more periods during a circadian cycle during which administration of the therapy is preferred to facilitate the transition of the circadian rhythm of the user, defining a transition circadian administration time;
determining the position of the transition circadian administration time within the normal circadian rhythm of the user, defining a user transition circadian administration time;
determining a time of day corresponding to the user transition circadian administration time responsive to the normal circadian rhythm of the user, defining a transition clock administration time; and
at least one of providing an indication to the user to administer the therapy at the transition clock administration time and administering the therapy to the user at the transition clock administration time.

7. The method of claim 1 further comprising receiving an indication of a present circadian phase of the user; wherein the clock administration time is determined responsive to the present circadian phase of the user.

8. The method of claim 7 wherein the present circadian phase is a first present circadian phase, the method further comprising:
receiving an indication of when the therapy was administered, defining an administration time;
determining an estimated circadian phase coinciding with the administration time;
determining an estimated efficacy of the therapy responsive to the estimated circadian phase;
determining a second user circadian administration time to administer a second administration of the therapy responsive to at least one of the estimated efficacy, the circadian administration time, the user circadian administration time, and a second indication of the present circadian phase of the user;
determining a second clock administration time responsive to the second user circadian administration time; and
at least one of providing an indication to the user to administer the second administration of the therapy at the second clock administration time and administering the second administration of the therapy to the user at the second clock administration time.

9. The method of claim 7 wherein the indication of the present circadian phase of the user and the normal circadian rhythm of the user is at least one of the user's sleep-wake cycles, light-dark exposure, chronotype, sex, age, present time zone, future time zone, present work shift, future work shift, heat rate, heart rate variability, core temperature, skin temperature, and a biological marker.

10. The method of claim 1 wherein:
the therapy is a first therapy;
the circadian administration time is a first therapy circadian administration time;
the user circadian administration time is a first therapy user circadian administration time;
the clock administration time is a first therapy clock administration time; and
the indication provided to the user to administer the therapy is a first indication;
the method further comprises:
receiving an indication of a second therapy to be administered;
identifying one or more periods during a circadian cycle during which administration of the second therapy is preferred responsive to the first therapy also being administered, defining a second therapy circadian administration time;
determining the position of the second circadian administration time within the normal circadian rhythm of the user, defining a second therapy user circadian administration time;
determining a time of day corresponding to the second therapy user circadian administration time, defining a second therapy clock administration time; and
at least one of providing a second indication to the user to administer the second therapy at the second therapy clock administration time and administering the second therapy to the user at the second therapy clock administration time;
wherein the first therapy circadian administration time is determined responsive to the second therapy also being administered.

11. The method of claim 10 further comprising receiving an indication of a present circadian phase of the user; wherein each of the first clock administration time and the second clock administration time is determined responsive to the present circadian phase of the user.

12. The method of claim 10 wherein:
providing the first indication to the user to administer the first therapy comprises at least one of illuminating a first indicator light, operating a sound-generating device to provide a first audio indication, operating a visual display to display a first message, and transmitting a first message to be received on a computerized device; and
providing the second indication to the user to administer the first therapy comprises at least one of illuminating a second indicator light that is differentiated from the first indicator light, operating a sound-generating device to provide a second audio indication that is differentiated from the second audio indication, operating a visual display to display a second message that is differentiated from the first message, and transmitting a second message that is differentiated from the first message to be received on the computerized device.

13. The method of claim 10 wherein
administering the first therapy to the user comprises at least one of operating a first machine to enable a first medication associated with the first therapy to be retrieved for administration and operating a first machine to administer the first therapy; and
administering the second therapy to the user comprises at least one of operating one of the first machine and a second machine to enable a second medication associated with the second therapy to be retrieved for administration, the second medication being differentiated from the first medication, and operating at least one of the first machine and a second machine to administer the second therapy that is differentiated from the first therapy.

14. A method of determining and administering therapies responsive to a circadian rhythm of a user comprising:
receiving an indication of the therapy to be administered to the user;
identifying one or more periods during a circadian cycle during which administration of the therapy is preferred, defining a circadian administration time;
receiving an indication of a present circadian phase of the user;
receiving an indication of a normal circadian rhythm of the user;
determining the position of the circadian administration time within the normal circadian rhythm of the user, defining a user circadian administration time;
determining a time of day corresponding to the user circadian administration time responsive to the present circadian phase of the user, defining a clock administration time; and
at least one of providing an indication to the user to administer the therapy at the clock administration time and administering the therapy to the user at the clock administration time.

15. The method of claim 14 wherein providing an indication to the user to administer the therapy comprises at least one of illuminating an indicator light, operating a sound-generating device to provide an audio indication, operating a visual display to display a message, and transmitting a message to be received on a computerized device.

16. The method of claim 14 wherein administering the therapy to the user at the clock administration time comprises at least one of dispensing a medication associated with the therapy such that the medication may be retrieved for administration and operating a machine to administer the therapy.

17. The method of claim 14 further comprising:
receiving an indication of when the therapy was administered, defining an administration time;
determining an estimated circadian phase coinciding with the administration time;
determining an estimated efficacy of the therapy responsive to the estimated circadian phase;
determining a second user circadian administration time to administer a second administration of the therapy responsive to at least one of the estimated efficacy, the circadian administration time, and the user circadian administration time;
determining a second clock administration time; and
at least one of providing an indication to the user to administer the second administration of the therapy at the second clock administration time and administering the second administration of the therapy to the user at the second clock administration time.

18. The method of claim 14 further comprising:
receiving an indication of an expected transition of the circadian rhythm of the user;
identifying one or more periods during a circadian cycle during which administration of the therapy is preferred to facilitate the transition of the circadian rhythm of the user, defining a transition circadian administration time;
determining the position of the transition circadian administration time within the normal circadian rhythm of the user, defining a user transition circadian administration time;
determining a time of day corresponding to the user transition circadian administration time responsive to the normal circadian rhythm of the user, defining a transition clock administration time; and
at least one of providing an indication to the user to administer the therapy at the transition clock administration time and administering the therapy to the user at the transition clock administration time.

19. The method of claim 14 wherein the present circadian phase is a first present circadian phase, the method further comprising:
receiving an indication of when the therapy was administered, defining an administration time;
determining an estimated circadian phase coinciding with the administration time;
determining an estimated efficacy of the therapy responsive to the estimated circadian phase;
determining a second user circadian administration time to administer a second administration of the therapy responsive to at least one of the estimated efficacy, the circadian administration time, the user circadian administration time, and a second indication of the present circadian phase of the user;
determining a second clock administration time responsive to the second user circadian administration time; and
at least one of providing an indication to the user to administer the second administration of the therapy at the second clock administration time and administering the second administration of the therapy to the user at the second clock administration time.

20. The method of claim 14 wherein the indication of the present circadian phase of the user and the normal circadian rhythm of the user is at least one of the user's sleep-wake cycles, light-dark exposure, chronotype, sex, age, present time zone, future time zone, present work shift, future work shift, heat rate, heart rate variability, core temperature, skin temperature, and a biological marker.

21. The method of claim 14 wherein:
the therapy is a first therapy;
the circadian administration time is a first therapy circadian administration time;
the user circadian administration time is a first therapy user circadian administration time;
the clock administration time is a first therapy clock administration time; and
the indication provided to the user to administer the therapy is a first indication;
the method further comprises:
receiving an indication of a second therapy to be administered;
identifying one or more periods during a circadian cycle during which administration of the second therapy is preferred responsive to the first therapy also being administered, defining a second therapy circadian administration time;
determining the position of the second circadian administration time within the normal circadian rhythm of the user, defining a second therapy user circadian administration time;
determining a time of day corresponding to the second therapy user circadian administration time, defining a second therapy clock administration time; and
at least one of providing a second indication to the user to administer the second therapy at the second therapy clock administration time and administering the second therapy to the user at the second therapy clock administration time;
wherein the first therapy circadian administration time is determined responsive to the second therapy also being administered.

22. The method of claim 21 further comprising receiving an indication of a present circadian phase of the user; wherein each of the first clock administration time and the second clock administration time is determined responsive to the present circadian phase of the user.

23. The method of claim 21 wherein:
providing the first indication to the user to administer the first therapy comprises at least one of illuminating a first indicator light, operating a sound-generating device to provide a first audio indication, operating a visual display to display a first message, and transmitting a first message to be received on a computerized device; and
providing the second indication to the user to administer the first therapy comprises at least one of illuminating a second indicator light that is differentiated from the first indicator light, operating a sound-generating device to provide a second audio indication that is differentiated from the second audio indication, operating a visual display to display a second message that is differentiated from the first message, and transmitting a second message that is differentiated from the first message to be received on the computerized device.

24. The method of claim 21 wherein
administering the first therapy to the user comprises at least one of operating a first machine to enable a first medication associated with the first therapy to be retrieved for administration and operating a first machine to administer the first therapy; and
administering the second therapy to the user comprises at least one of operating one of the first machine and a second machine to enable a second medication associated with the second therapy to be retrieved for administration, the second medication being differentiated from the first medication, and operating at least one of the first machine and a second machine to administer the second therapy that is differentiated from the first therapy.

25. A system for determining and facilitating administration of therapies responsive to a circadian rhythm of a patient comprising:
a network communication device configured to:
receive an indication of a therapy to be administered to the patient;
receiving an indication of a normal circadian rhythm of the patient; and
transmit a signal to a remote computerized device configured to at least one of provide an indication to administer the therapy at a clock administration time and administer the therapy at the clock administration time;
a processor positioned in communication with the network communication device and configured to:
identify one or more periods during a circadian cycle during which administration of the therapy is preferred, defining a circadian administration time;
determine the position of the circadian administration time within the normal circadian rhythm of the user, defining a user circadian administration time; and
determine a time of day corresponding to the user circadian administration time, defining a clock administration time; and
a non-transitory storage medium positioned in communication with each of the processor and the network communication device.
